(19) United States
(12) Patent Application Publication
(10) Pub. No.: US 2017/0156914 A1
(43) Pub. Date: Jun. 8, 2017

(54) Neck Relief Arm Sling

(71) Applicant: Therapeutic Dimensions, Inc., Spokane, WA (US)

(72) Inventor: James Talley Abel, Spokane, WA (US)

(73) Assignee: Therapeutic Dimensions, Inc., Spokane, WA (US)

(21) Appl. No.: 14/963,221

(22) Filed: Dec. 8, 2015

(51) Int. Cl. A61F 5/37

(52) U.S. Cl. CPC A61F 5/3738

(57) ABSTRACT

Representative implementations of devices and techniques provide an under-the-arm support for a sling device. The under-the-arm support includes a strap portion that is wrapped around a portion of a user's torso, including under the user's arm. One or more slide mechanisms are associated with the strap portion. The slide mechanism(s) are slideably coupled to a shoulder strap of the sling device, and have freedom to move with respect to the shoulder strap.

### BACKGROUND

Medical devices such as arm slings and the like may be used to support, immobilize, or restrict movement of an arm, for example. Arm slings may be worn by persons with wrist, arm, or shoulder injuries, for instance, to help prevent further injury and to promote healing. Using an arm sling can also help manage the pain associated with an injury by supporting the weight of the injured or associated arm.

Most arm slings and like devices include a pocket portion and a shoulder/neck strap. The injured arm (or the arm associated with the injured shoulder, wrist, etc.) is cradled in the pocket portion, which generally comprises a cloth pouch or a similar device. The shoulder/neck strap is commonly attached to the pocket portion at two points—near the wrist/hand opening of the pocket portion and near the elbow area of the pocket portion. The shoulder/neck strap is looped around the neck of the user, to an opposite side of the user's neck from the slinged arm, such that the weight of the slinged arm within the pocket portion is initially supported by the shoulder or the neck of the user.

In such an arrangement, the force applied by the weight of the slinged arm is directed downward and inward (toward the pocket portion and toward the user's neck). Accordingly, even when the shoulder/neck strap is initially placed closer to the user's shoulder, the inward pulling force applied by the weight of the slinged arm tends to move the shoulder/neck strap closer to the user's neck as the sling is worn over a short period of time. This shoulder/neck strap movement shifts increasingly more of the weight of the slinged arm to the user's neck, and can cause the user neck pain, neck injury, and related discomfort.

In some cases, a shoulder/neck strap may be padded to improve the comfort of wearing the arm sling over time. For example, soft padding can be located on the shoulder/neck strap and positioned at the user's neck. However, while the soft padding may reduce some discomfort associated with the shoulder/neck strap rubbing on the user's neck (causing chafing, abrasions, etc. of the skin on the user's neck), the soft padding does not reduce the amount of force applied to the user's neck by the weight of the slinged arm. Consequently, neck pain, fatigue, headaches, and neck injury may still occur with prolonged use of the arm sling.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

For this discussion, the devices and systems illustrated in the figures are shown as having a multiplicity of components. Various implementations of devices and/or systems, as described herein, may include fewer components and remain within the scope of the disclosure. Alternately, other implementations of devices and/or systems may include additional components, or various combinations of the described components, and remain within the scope of the disclosure. Shapes and/or dimensions shown in the illustrations of the figures are for example, and other shapes and or dimensions may be used and remain within the scope of the disclosure, unless specified otherwise.

FIG. 1 is a front view of a user wearing an example sling device including an under-the-arm (UTA) support strap, according to an embodiment.

FIG. 2 is a back view of a user wearing an example sling device including a UTA strap, according to an embodiment.

FIG. 3 includes two front views showing an example application of a UTA strap, according to an embodiment.

FIG. 4 includes two front views showing another example application of a UTA strap, according to an embodiment.

FIG. 5 includes two perspective views of example UTA straps, according to two embodiments.

FIG. 6 includes a front view and a back view of an example application of a UTA strap, according to another embodiment.

FIG. 7 is a flow diagram of an example process of shifting the weight of an arm in an arm sling, using a UTA strap, according to an embodiment.

### DETAILED DESCRIPTION

### Introduction

Representative implementations of devices and techniques provide an under-the-arm (UTA) support strap for a sling device, such as an arm sling. The under-the-arm support strap includes a strap portion that is wrapped around a portion of a user's torso, including under the user's arm. One or more slide mechanisms are associated with the strap portion. In one embodiment, a slide mechanism is attached to each end of the strap portion. The slide mechanism(s) are slideably coupled to a shoulder strap (for instance) of the sling device, and have freedom to move with respect to the shoulder strap.

In various implementations, the UTA support strap positions the shoulder strap of the sling device on the user, and maintains the shoulder strap in a desired location and position. For example, the UTA support strap can maintain the shoulder strap at a preferred location on the user's shoulder, even while the sling device is worn over a prolonged period of time. Positioning and maintaining the shoulder strap in the desired location and position can shift the force applied by the weight of the slinged arm (i.e., load) away from the user's neck and toward the user's shoulder.

In various embodiments, the load of the arm sling may be shifted laterally, away from the user's neck and toward a shoulder of the user, by sliding one or more of the slide mechanisms with respect to the shoulder strap. In other words, one or more of the slide mechanisms may be moved upwards or downwards on the shoulder strap to reposition or adjust the shoulder strap on the user, shifting the load of the slinged arm on the user, and reducing a force applied to the user's neck from the shoulder strap.

In an implementation, the strap portion includes one or more adjustment components. For example a length of the strap portion may be adjusted via the one or more adjustment components. The user may desire to adjust the length of the strap portion for better fit, or for positioning and maintaining the shoulder strap in the desired location and position. For example, the length of the strap portion may be adjusted to shift the force applied by the weight of the slinged arm away from the user's neck and toward the user's shoulder.

In various embodiments, the load of the arm sling may be shifted laterally, away from the user's neck and toward a shoulder of the user, by adjusting a length of the strap portion. In other words, one or more of the adjustment components may be adjusted to lengthen or shorten a length of the UTA support strap, to reposition or adjust the shoulder strap on the user, shifting the load of the slinged arm on the user, and reducing a force applied to the user's neck from the shoulder strap.

In some implementations, the adjustment components are integral to or associated with the one or more slide mechanisms. In other implementations, the adjustment components are separate from the one or more slide mechanisms. In one example, the slide mechanisms may incorporate the adjustment components. For instance, in an implementation, one or more of the slide mechanisms comprise a strip of material looped around the shoulder strap and coupled to itself via hook and loop fasteners, snaps, or other closure features. In such a case, the adjustment components may include the hook and loop fasteners, multiple snap locations, or the like.

In another example, the slide mechanisms may be coupled to the strap portion via the adjustment components. For instance, the slide mechanisms may be attached to the strap portion using loops, buckles, d-rings, etc. that provide length adjustment to the strap portion as well as attach the slide mechanisms to the strap portion.

In various implementations, the strap portion is comprised of multiple layers. The layers may be combined to provide various desired characteristics for the strap portion, such as strength, comfort, resilience, utility, weight, and so forth. For instance, outer layers may provide strength and utility while inner layers may provide comfort.

Advantages of the disclosed devices and techniques are varied, and include: better stabilization of the load (e.g., weight) of the slinged arm; improved weight distribution of the load over the user's shoulder and off the user's neck; beneficial comfort in wearing a sling, particularly over a longer period of time; and a highly functional but inexpensive solution, making it accessible to greater numbers of patients. Other advantages may also be appreciated by one skilled in the art.

Techniques and devices are discussed with reference to example arm slings illustrated in the figures. However, this is not intended to be limiting, and is for ease of discussion and illustrative convenience. The techniques and devices discussed may be applied to various sling devices, and the like, arranged to be worn in any of various arrangements, for supporting, stabilizing, or otherwise restricting a user's limb, and remain within the scope of the disclosure. In alternate implementations, the techniques and devices disclosed may be employed in other ways or with other devices, systems, arrangements, and so forth.

Implementations are explained in more detail below using a plurality of examples. Although various implementations and examples are discussed here and below, further implementations and examples may be possible by combining the features and elements of individual implementations and examples.

### Example Sling Device

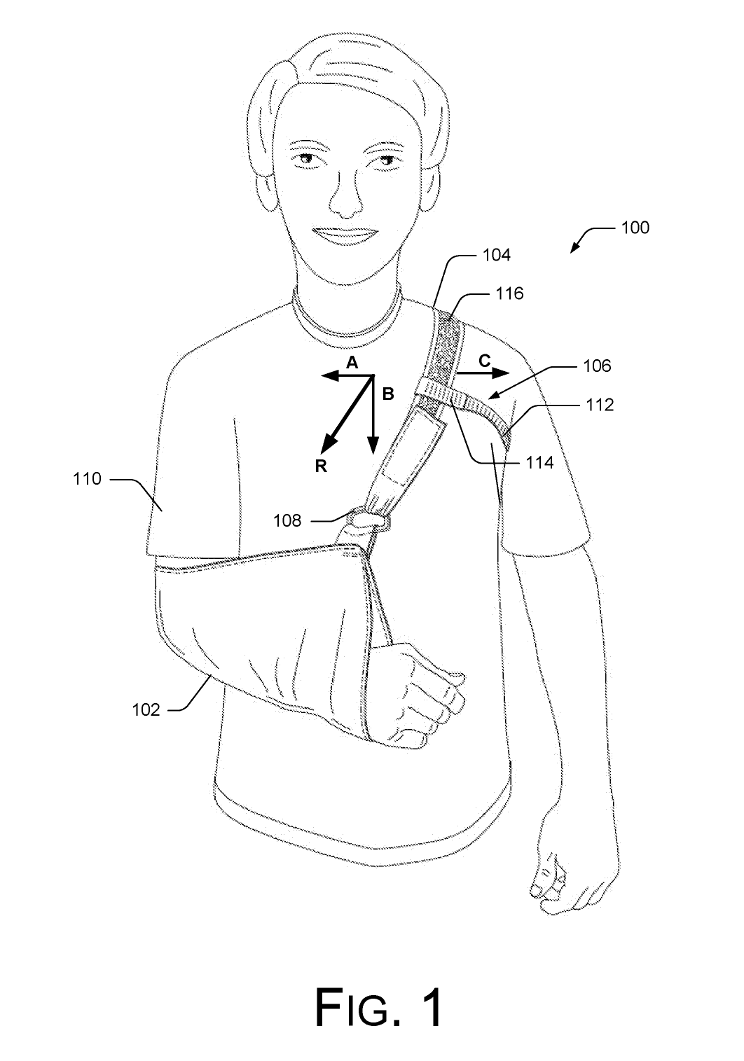

Referring to FIGS. 1 through 6, an example sling device 100 is shown in several non-limiting example configurations. In various embodiments, as shown in the FIGS. 1-6, a sling device 100 includes a pocket portion 102, a shoulder strap 104, and an under-the-arm (UTA) support strap 106. In alternate embodiments, the sling device 100 may also include one or more connectors 108 for coupling the pocket portion 102 to the shoulder strap 104. In some examples, the one or more connectors 108 may provide or facilitate adjustment of the shoulder strap 104.

In various implementations, the pocket portion 102 is arranged to cradle a user's arm 110. For example, at least a portion of the user's arm 110 is placed within or is supported by the pocket portion 102. In the implementations, the pocket portion 102 may comprise a pouch, one or more straps, a tube, a sheet of material, or the like.

In an implementation, the shoulder strap 104 is coupled to the pocket portion 102 at one or more locations. In various examples, each end of the shoulder strap 104 is coupled to the pocket portion 102. In one example, a first end of the shoulder strap 104 is coupled to the pocket portion 102 near the wrist/hand opening of the pocket portion 102 and a second end of the shoulder strap 104 is coupled to the pocket portion 104 near the elbow area of the pocket portion 104. In alternate examples, the shoulder strap 104 may be coupled to the pocket portion 102 in various other locations. In each of the examples, the shoulder strap 104 is positioned over the user's shoulder to support a weight of the user's arm 110.

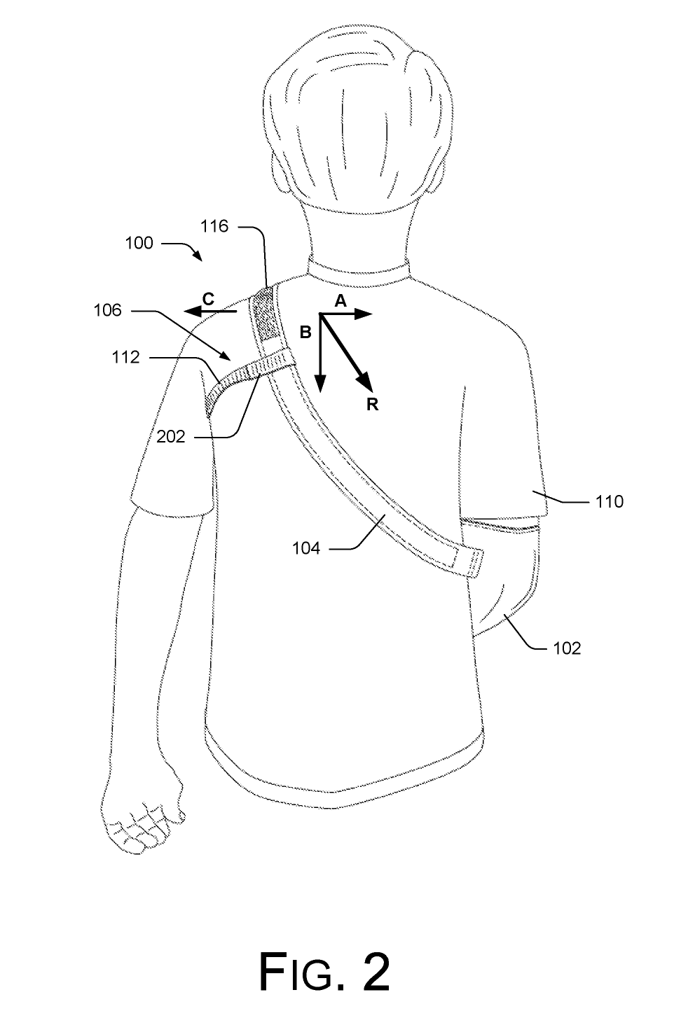

In such an arrangement, as shown in FIGS. 1 and 2, the force (R) applied by the weight of the slinged arm 110 on the shoulder strap 104 has a downward component (direction B) and an inward component (direction A) toward the user's neck. Accordingly, the inward pulling force (A) applied by the weight of the slinged arm 110 can tend to move the shoulder strap 104 closer to the user's neck as the sling 100 is worn over a short period of time. This shoulder strap 104 movement shifts increasingly more of the weight of the slinged arm 110 to the user's neck, and can cause the user neck pain, neck injury, and related discomfort.

In various embodiments, the UTA support strap 106 is arranged to be wrapped around a portion of the user's torso, including under an arm of the user. The UTA support strap 106 is coupled at one or more points to the shoulder strap 104. As shown in FIGS. 1 and 2, the UTA support strap 106 applies an outward lateral force (C) that reduces or eliminates the inward component (A) of the force (R) applied by the weight of the slinged arm 110 on the shoulder strap 104. Thus, the UTA support strap 106 positions and maintains the shoulder strap 104, and thus the downward component (B) of the weight of the slinged arm 110, at a preferred or desired location (on the user's shoulder, for example). Positioning and maintaining the shoulder strap 104 in the desired location and position can shift the load (i.e., the force (B) or (R)) of the slinged arm 110 off of and away from the user's neck.

In an implementation, the UTA support strap 106 includes a strap portion 112 and one or more slide mechanisms 114. In various embodiments, the strap portion 112 is arranged to be wrapped around a portion of a user's torso, including under an arm of the user. In an embodiment, the strap portion 112 is approximately 18 inches in length. In other embodiments, the strap portion 112 is longer or shorter, to accommodate the torso of the user.

In some embodiments, the strap portion 112 may be comprised of multiple layers. The layers may be combined to provide various desired characteristics for the strap portion 112, such as strength, comfort, resilience, utility, weight, and so forth. For instance, outer layers may provide strength and utility while inner layers may provide comfort.

In various embodiments, the strap portion 112 comprises one or more resilient elastic or semi-elastic materials (e.g., spandex, rubber, synthetic rubber, polymers, or the like), one or more non-elastic materials (e.g., a natural fiber material, a nylon material, a para-aramid or aramid material, a fiberglass material, a metallic material, an animal hide, or the like), or combinations of both. For example, the strap portion 112 may be generally elastic, to apply the outward lateral force (C) on the shoulder strap 104. In other examples, the strap portion 112 may be generally non-elastic, and may apply the outward lateral force (C) by pushing and/or pulling on the shoulder strap 104. In some alternate embodiments, the strap portion 112 comprises a semi-flexible or non-flexible material (e.g., plastic, composite, fiberglass, metal, etc.), forming a semi-rigid or rigid band or strap. For example, in the embodiments, the strap portion 112 may be molded, stamped, or otherwise formed. In such examples, the strap portion 112 may also apply the outward lateral force (C) by pushing and/or pulling on the shoulder strap 104.

In various implementations, the UTA support strap 106 includes a first slide mechanism 114 coupled to a first end of the strap portion 112. In the implementations, the first slide mechanism 114 is slideably coupled to a first portion (e.g., the front portion) of the shoulder strap 104 of the sling 100, such that the first slide mechanism 114 has freedom to slide with respect to the shoulder strap 104.

In some implementations, the UTA support strap 106 includes a second slide mechanism 202 coupled to a second end of the strap portion 112. In the implementations, the second slide mechanism 202 is slideably coupled to a second portion (e.g., the back portion) of the shoulder strap 104 of the sling 100, such that the second slide mechanism 202 has freedom to slide with respect to the shoulder strap 104.

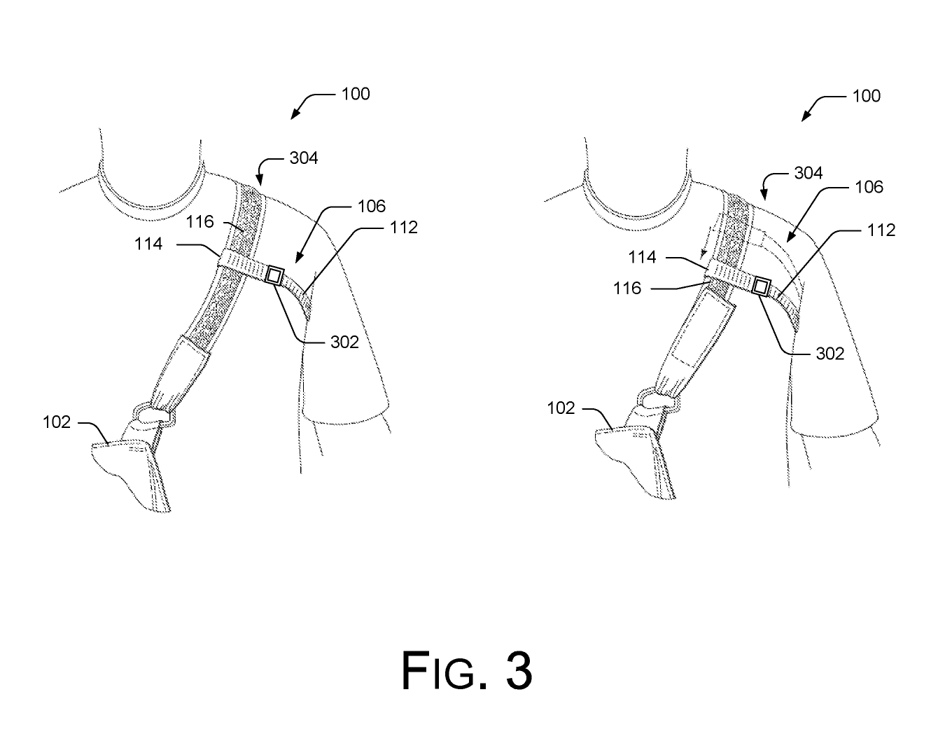

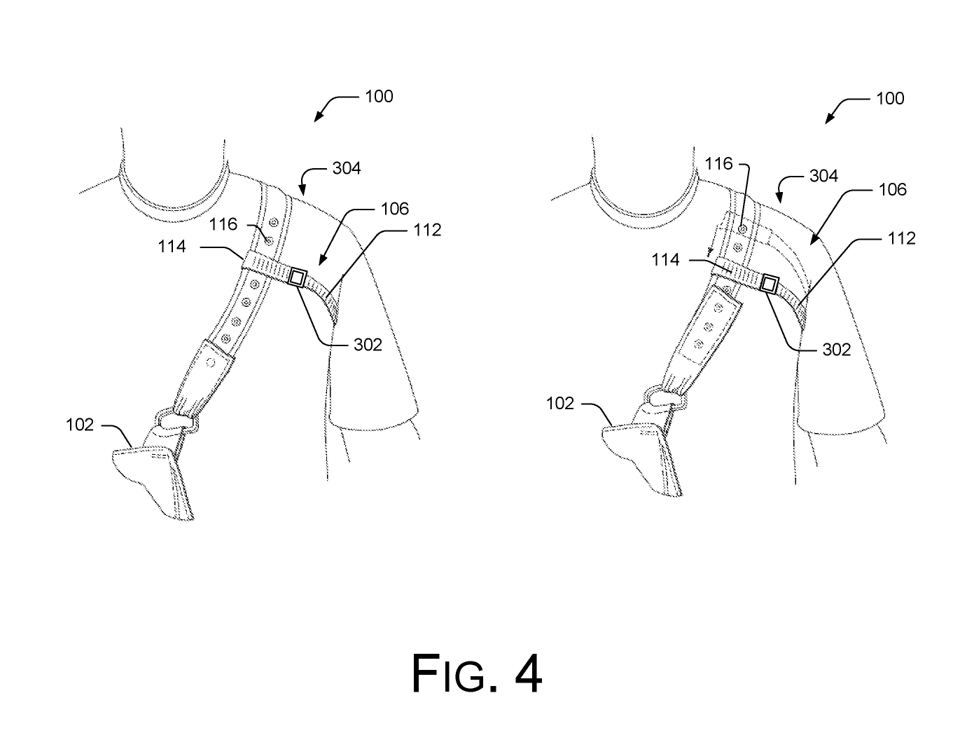

In various embodiments, as shown in FIGS. 3 and 4, the UTA support strap 106 shifts the load ((R) or (B)) of the arm sling laterally, away from the user's neck and toward a shoulder of the user (in the direction (C)), by sliding the first 114 and/or second 202 slide mechanisms with respect to the shoulder strap 104. In the embodiments, sliding the first 114 and/or second 202 slide mechanisms with respect to the shoulder strap 104 reduces or eliminates a force ((R) or (B)) applied to the user's neck from the shoulder strap 104. For instance, sliding the first 114 and/or second 202 slide mechanisms with respect to the shoulder strap 104 can position and/or re-position (i.e., adjust) the shoulder strap 104 on the user (in the front and/or the back of the user), shifting the load of the slinged arm 110.

In an implementation, as shown in FIGS. 1-4, the first 114 and/or second 202 slide mechanisms may be held in place on the shoulder strap 104 via a temporary fastener 116. For example, once the first 114 and/or second 202 slide mechanisms are positioned, re-positioned, or adjusted, the first 114 and/or second 202 slide mechanisms may be temporarily fixed in place with the temporary fastener 116. In various embodiments, the temporary fastener 116 comprises hook and loop fastener, snaps, hooks, buttons, or the like. Accordingly, one or more surfaces (e.g., an inside surface, for example) of the first 114 and/or second 202 slide mechanisms may include a portion of the temporary fastener 116 (e.g., the hook or loop material, one half of a snap device, a catch for a hook device, a button hole, etc.).

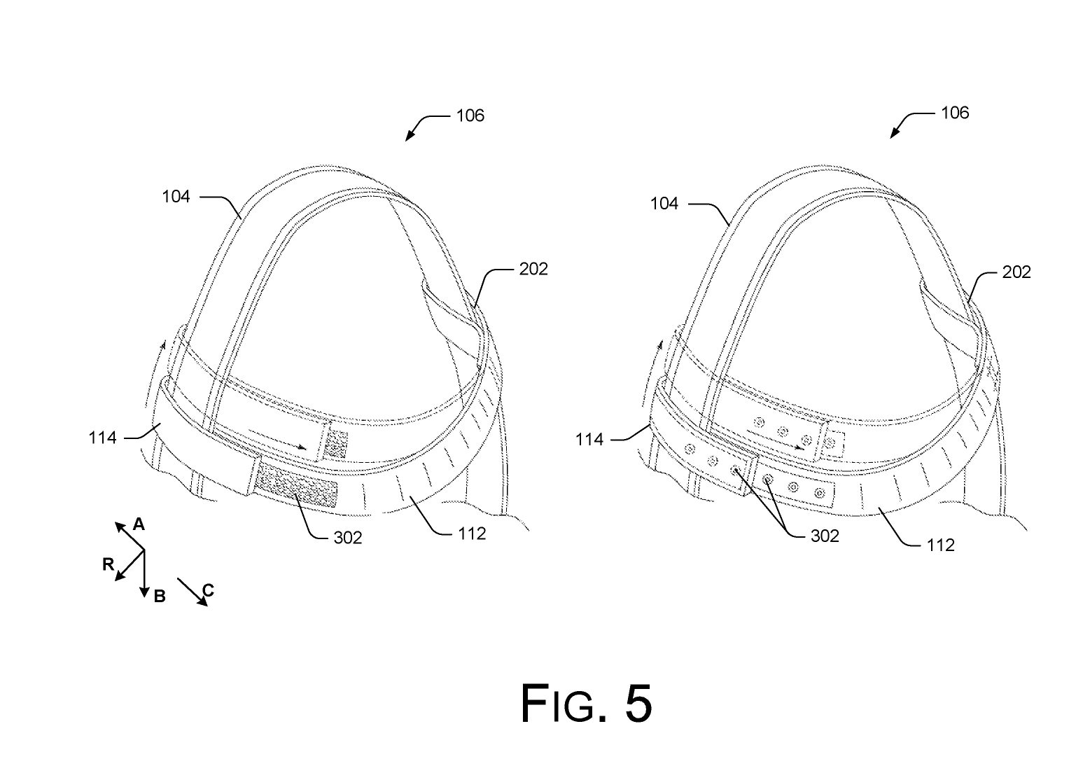

In an embodiment, the first slide mechanism 114 and/or the second slide mechanism 202 comprises a loop or tube. In one example, the first 114 and/or second 202 slide mechanisms comprise an extension of the material of the strap portion 112. In various examples, as shown in FIG. 5, the first 114 and/or second 202 slide mechanisms may comprise an extension of the strap portion 112 (using the same material or other materials) that is looped around the shoulder strap 104, and coupled back on itself

In another embodiment, the first slide mechanism 114 and/or the second slide mechanism 202 comprises a loop or tube comprised of a material such as metal, plastic, fabric, animal hide, or the like. In further embodiments, the first slide mechanism 114 and/or the second slide mechanism 202 comprises an elliptical ring, a d-ring, a polygonal ring, a strip of material having a hook and loop fastener thereon, or various other components, allowing the first slide mechanism 114 and/or the second slide mechanism 202 to slide with respect to the shoulder strap 104.

In an embodiment, the first slide mechanism 114 and the second slide mechanism 202 comprise a single tube (such as a shoulder pad wrapped around the shoulder strap 104, or the like), slideably coupled to the shoulder strap 104, such that the shoulder pad has freedom to slide with respect to the shoulder strap 104. In other words, the first 114 and second 202 slide mechanisms comprise the same component or two components joined to form a single component. For example, the first end of the strap portion 112 is coupled to a first end of the tube and the second end of the strap portion 112 is coupled to a second end of the tube. In such an embodiment, the first 114 and second 202 slide mechanisms are positioned in substantially the same location with respect to the shoulder strap 104.

In the embodiment, the UTA support strap 106 shifts the load ((R) or (B)) of the slinged arm 110 laterally, away from the user's neck and toward a shoulder of the user (in the direction (C)), by sliding the first 114 and second 202 slide mechanisms (as a single component) with respect to the shoulder strap 104. This reduces or eliminates the force ((R) or (B)) applied to the user's neck from the shoulder strap 104 by positioning and/or re-positioning (i.e., adjust) the shoulder strap 104 on the user.

In various implementations, as shown in FIGS. 3-5, the UTA support strap 106 includes a first adjustment component 302 coupling the first slide mechanism 114 to the first end of the strap portion 112. In the implementations, the first adjustment component 302 is arranged to change a length of the UTA support strap 106. In some implementations, the UTA support strap 106 includes a second adjustment component (304) coupling the second slide mechanism 202 to the second end of the strap portion 112. In the implementations, the second adjustment component 304 may also be adjusted to change a length of the UTA support strap 106. In an alternate implementation, the UTA support strap 106 includes one or more adjustment components (not shown) at a location along the length of the strap portion 112. In the implementations, the one or more adjustment components may also be adjusted to change a length of the UTA support strap 106.

In various embodiments, as shown in FIG. 5, the UTA support strap 106 shifts the load ((R) or (B)) of the arm sling laterally, away from the user's neck and toward a shoulder of the user (in the direction (C)), by adjusting a length of the UTA strap 106. In the embodiments, adjusting the first 302 and/or second 304 adjustment components reduces or eliminates a force ((R) or (B)) applied to the user's neck from the shoulder strap 104. For instance, adjusting the first 302 and/or second 304 adjustment components can position and/or re-position (i.e., adjust) the shoulder strap 104 on the user (in the front and/or the back of the user), shifting the load of the slinged arm 110. In an implementation, as shown in FIG. 5, the UTA support strap 106 shifts the load ((R) or (B)) of the arm sling laterally, away from the user's neck and toward a shoulder of the user (in the direction (C)), and reduces or eliminates a force ((R) or (B)) applied to the user's neck from the shoulder strap 104, by sliding the first 114 and/or second 202 slide mechanisms with respect to the shoulder strap 104 and adjusting a length of the UTA strap 106.

In an embodiment, the first adjustment component 302 and/or second adjustment component 304 comprise a hook and loop fastener, a buckle, a d-ring, a pair of cinch rings, a snap device, or various other components, allowing the user to shorten or lengthen a length of the UTA support strap 106. In another embodiment, the first 302 or second 304 adjustment components may be coupled to an end of the first 114 and/or second 202 slide mechanisms, and particularly when the first 114 and second 202 slide mechanisms are coupled together as a single component, as described above. For example, the strap portion 112 may be threaded through a d-ring (or the like) attached to one or both ends of the single component, and looped back on itself to adjust a length of the strap portion 112.

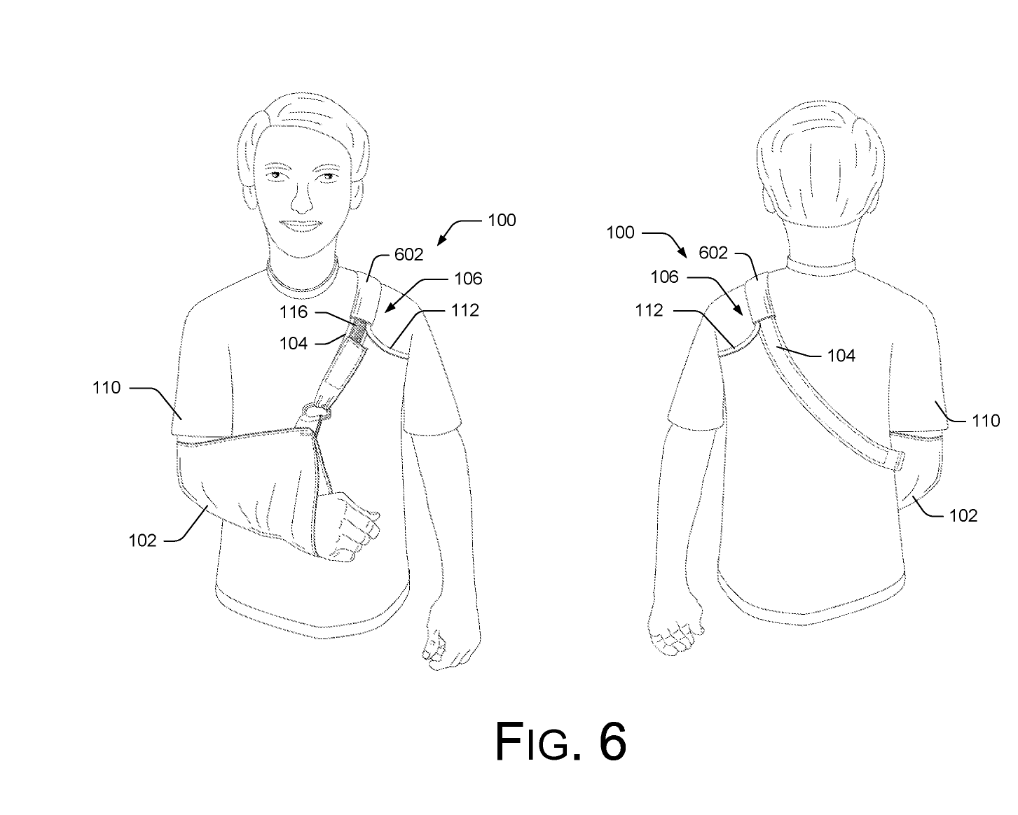

FIG. 6 includes a front view and a back view of an example alternate application of a UTA support strap 106, according to another embodiment. In the embodiment, the first slide mechanism 114 and the second slide mechanism 202 comprise a single slide mechanism 602 (such as a shoulder pad wrapped around the shoulder strap 104, or the like), slideably coupled to the shoulder strap 104, such that the slide mechanism 602 has freedom to slide with respect to the shoulder strap 104.

As shown in FIG. 6, the strap portion 112 of the UTA support strap 106 is wrapped around a portion of the user's torso, including under an arm of the user, and through the slide mechanism 602. In various examples, the ends of the strap portion 112 may be coupled together, and may be coupled to the inside of the slide mechanism 602.

Additionally, the UTA support strap 106 may include one or more adjustment components coupled to the UTA strap and arranged to change a length of the UTA strap, as described above. For instance, the one or more adjustment components may include hook and loop fastener, a buckle, a d-ring, a pair of cinch rings, a snap, or the like. In an example, an adjustment component may be used to couple the ends of the strap portion 112 together, adjusting a length of the strap portion 112.

The examples shown in FIGS. 1-6 and described herein are not intended to be limiting. In alternate implementations, UTA support strap 106 or the sling device 100 may have other arrangements or configurations, and may be used with additional components to reduce or eliminate forces on the neck of the user when the UTA support strap 106 is wrapped around the user.

The components and techniques discussed herein with respect to the UTA support strap 106 are intended to be used in the production of new UTA support straps 106 or sling devices 100 or in the retro-fitting of existing sling devices. In various embodiments, existing sling devices may be upgraded or re-fitted with one or more of the components (e.g., a UTA support strap 106), and/or using one or more of the described techniques either individually or in various combinations. In an embodiment, the described components and techniques result in a UTA support strap 106 capable of interchangeably coupling to various types of sling devices, or the like. Further, such modified sling devices may be configured and/or adjusted for various user wearing options and locations.

In various implementations, additional or alternative components, techniques, sequences, or processes may be used to implement the techniques described herein. Further, the components and/or techniques may be arranged and/or combined in various combinations, while resulting in similar or approximately identical results. It is to be understood that a UTA support strap 106 may be implemented as a stand-alone device or as part of a system (e.g., integrated with other components to form a sling device 100, as described above).

### Representative Process

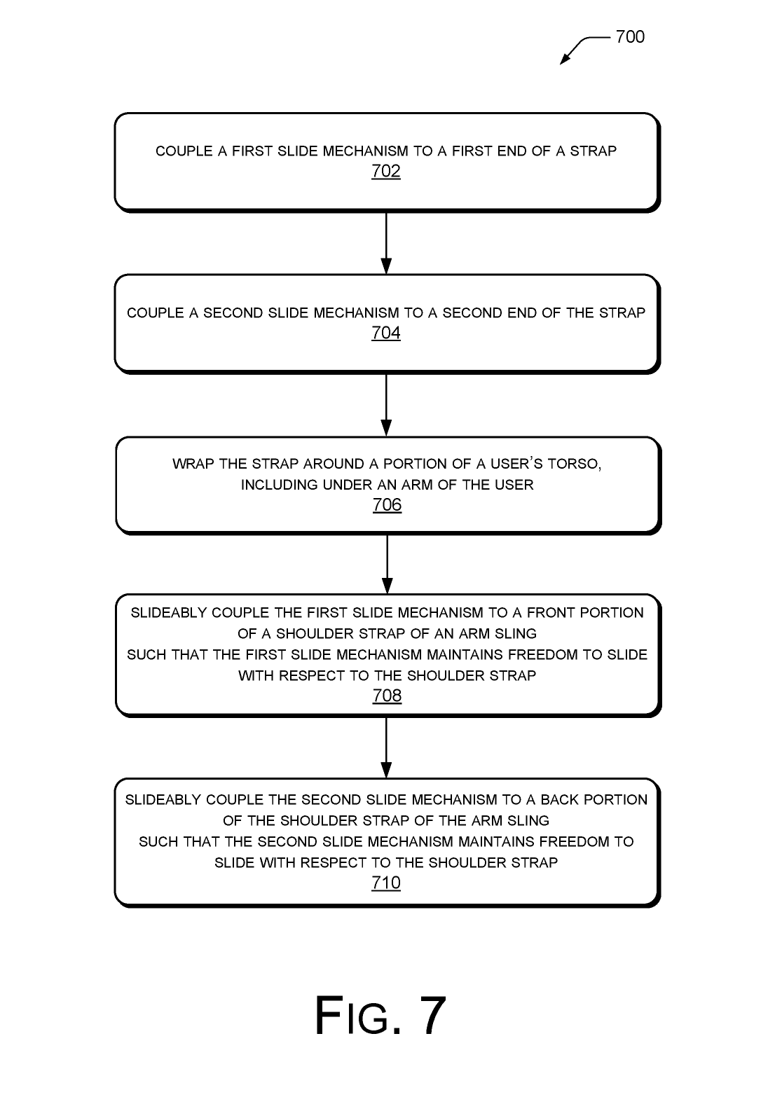

FIG. 7 is a flow diagram illustrating an example method 700 for shifting the weight of an arm in a sling (such as sling 100, for example) using an under-the-arm (UTA) support strap (such as UTA support strap 106, for example), according to various implementations. The process 700 is described with reference to FIGS. 1-6.

The order in which the process is described is not intended to be construed as a limitation, and any number of the described process blocks can be combined in any order to implement the process, or alternate processes. Additionally, individual blocks may be deleted from the process without departing from the spirit and scope of the subject matter described herein. Furthermore, the process can be implemented in any suitable materials, or combinations thereof, without departing from the scope of the subject matter described herein.

At block 702, the process includes coupling a first slide mechanism (such as first slide mechanism 114, for example) to a first end of a strap (such as strap portion 112, for example). In one implementation, attaching the first slide mechanism to the strap forms a UTA support strap. In another implementation, a second slide mechanism is also needed to form a UTA support strap. At block 704, the process includes coupling a second slide mechanism (such as second slide mechanism 202, for example) to a second end of the strap.

In an implementation, the process includes coupling the first slide mechanism to the first end of the strap via a first adjustment component (such as first adjustment component 302, for example) and coupling the second slide mechanism (if it is present) to the second end of the strap via a second adjustment component (such as second adjustment component 304, for example). In the implementation, the first and second adjustment components are arranged to change a length of the strap.

At block 706, the process includes wrapping the strap around a portion of a user's torso, including under an arm of the user. At block 708, the process includes slideably coupling the first slide mechanism to a front portion of a shoulder strap (such as shoulder strap 104, for example) of an arm sling such that the first slide mechanism maintains freedom to slide with respect to the shoulder strap. At block 710, the process includes slideably coupling the second slide mechanism to a back portion of the shoulder strap of the arm sling such that the second slide mechanism maintains freedom to slide with respect to the shoulder strap.

In one implementation, the process includes shifting a load of the arm sling laterally, away from the user's neck and toward a shoulder of the user, by sliding the first and/or second slide mechanisms with respect to the shoulder strap. In another implementation, the process includes shifting a load of the arm sling laterally, away from the user's neck and toward a shoulder of the user, by adjusting a length of the UTA support strap.

In one implementation, the process includes reducing a force applied to the user's neck from the shoulder strap, by sliding the first and/or second slide mechanisms with respect to the shoulder strap. In another implementation, the process includes reducing a force applied to the user's neck from the shoulder strap, by adjusting a length of the UTA support strap.

In an implementation, the process includes inserting the shoulder strap through a tube. In one example, the process includes coupling the first end of the UTA support strap to a first end of the tube and coupling the second end of the UTA support strap to a second end of the tube. In another example, the process includes coupling the first and second ends of the UTA support strap to each other. In an embodiment, the process includes coupling the first and second ends of the UTA support strap to a portion of the tube (an inside surface of the tube, for example).

In alternate implementations, other techniques may be included in the process 700 in various combinations, and remain within the scope of the disclosure.

### CONCLUSION

While various discreet embodiments have been described throughout, the individual features of the various embodiments may be combined to form other embodiments not specifically described. The embodiments formed by combining the features of described embodiments are also within the scope of the disclosure.

1. An apparatus, comprising:
a strap portion arranged to be wrapped around a portion of a user's torso, including under an arm of the user;
a first slide mechanism coupled to a first end of the strap portion, the first slide mechanism arranged to be slideably coupled to a first portion of a shoulder strap of an arm sling such that the first slide mechanism has freedom to slide with respect to the shoulder strap; and
a second slide mechanism coupled to a second end of the strap portion, the second slide mechanism arranged to be slideably coupled to a second portion of the shoulder strap of the arm sling such that the second slide mechanism has freedom to slide with respect to the shoulder strap.

2. The apparatus of claim 1, further comprising a first adjustment component coupling the first slide mechanism to the first end of the strap portion, the first adjustment component arranged to change a length of the strap portion.

3. The apparatus of claim 2, further comprising a second adjustment component coupling the second slide mechanism to the second end of the strap portion, the second adjustment component arranged to change a length of the strap portion.

4. The apparatus of claim 3, wherein the first adjustment component and/or the second adjustment component are selected from the group comprising: a hook and loop fastener, a buckle, a d-ring, a pair of cinch rings, and a snap.

5. The apparatus of claim 1, wherein the first slide mechanism and/or the second slide mechanism comprises a tube.

6. The apparatus of claim 5, wherein the first slide mechanism and the second slide mechanism comprise a single tube, and wherein the first end of the strap portion is coupled to a first end of the tube and the second end of the strap portion is coupled to a second end of the tube.

7. The apparatus of claim 1, wherein the strap portion is comprised of a resilient elastic or semi-elastic material.

8. The apparatus of claim 1, wherein the strap portion is comprised of a resilient non-elastic material.

9. An arm sling assembly, comprising:
a pocket portion to cradle a user's arm;
a shoulder strap coupled at first and second ends of the shoulder strap to the pocket portion, and positioned over the user's shoulder to support a weight of the user's arm;
an under-the-arm (uta) strap arranged to be wrapped around a portion of the user's torso, including under an arm of the user;
a first slide mechanism coupled to a first end of the uta strap and slideably coupled to a first portion of the shoulder strap such that the first slide mechanism has freedom to slide with respect to the shoulder strap; and
a second slide mechanism coupled to a second end of the uta strap and slideably coupled to a second portion of the shoulder strap such that the second slide mechanism has freedom to slide with respect to the shoulder strap.

10. The arm sling assembly of claim 9, further comprising a first adjustment component coupling the first slide mechanism to the first end of the uta strap, the first adjustment component arranged to change a length of the uta strap.

11. The arm sling assembly of claim 10, further comprising a second adjustment component coupling the second slide mechanism to the second end of the uta strap, the second adjustment component arranged to change a length of the uta strap.

12. The arm sling assembly of claim 9, further comprising a shoulder pad slideably coupled to the shoulder strap such that the shoulder pad has freedom to slide with respect to the shoulder strap, the shoulder pad comprising a fabric tube.

13. The arm sling assembly of claim 12, wherein the shoulder pad is comprised of the first slide mechanism and the second slide mechanism in the form of a single component, and wherein the first end of the uta strap is coupled to a first end of the shoulder pad and the second end of the uta strap is coupled to a second end of the shoulder pad.

14. The arm sling assembly of claim 9, wherein the first slide mechanism and/or the second slide mechanism comprises a tube comprised of a material selected from the group including: metal, plastic, fabric, and animal hide.

15. The arm sling assembly of claim 9, wherein the first slide mechanism and/or the second slide mechanism is selected from the group including: a tube, a loop, an elliptical ring, a d-ring, a polygonal ring, and a strip of material having a hook and loop fastener thereon.

16. A method, comprising:
coupling a first slide mechanism to a first end of a strap;
coupling a second slide mechanism to a second end of the strap;
wrapping the strap around a portion of a user's torso, including under an arm of the user;
slideably coupling the first slide mechanism to a front portion of a shoulder strap of an arm sling such that the first slide mechanism maintains freedom to slide with respect to the shoulder strap; and
slideably coupling the second slide mechanism to a back portion of the shoulder strap of the arm sling such that the second slide mechanism maintains freedom to slide with respect to the shoulder strap.

17. The method of claim 16, further comprising inserting the shoulder strap through a tube and coupling the first end of the strap to a first end of the tube and coupling the second end of the strap to a second end of the tube.

18. The method of claim 16, further comprising coupling the first slide mechanism to the first end of the strap via a first adjustment component and coupling the second slide mechanism to the second end of the strap via a second adjustment component, the first and second adjustment components arranged to change a length of the strap.

19. The method of claim 16, further comprising shifting a load of the arm sling laterally, away from the user's neck and toward a shoulder of the user, by sliding the first and/or second slide mechanisms with respect to the shoulder strap.

20. The method of claim 16, further comprising shifting a load of the arm sling laterally, away from the user's neck and toward a shoulder of the user, by adjusting a length of the strap.

21. The method of claim 16, further comprising reducing a force applied to the user's neck from the shoulder strap, by sliding the first and/or second slide mechanisms with respect to the shoulder strap.

22. The method of claim 16, further comprising reducing a force applied to the user's neck from the shoulder strap, by adjusting a length of the strap.

23. An arm sling assembly, comprising:
a pocket portion to cradle a user's arm;
a shoulder strap coupled at first and second ends of the shoulder strap to the pocket portion, and positioned over the user's shoulder to support a weight of the user's arm;
a slide mechanism slideably coupled to the shoulder strap such that the slide mechanism has freedom to slide with respect to the shoulder strap; and
an under-the-arm (uta) strap arranged to be wrapped around a portion of the user's torso, including under an arm of the user, and through the slide mechanism.

24. The arm sling assembly of claim 23, further comprising one or more adjustment components coupled to the uta strap and arranged to change a length of the uta strap, the one or more adjustment components selected from the group comprising: a hook and loop fastener, a buckle, a d-ring, a pair of cinch rings, and a snap.
